(19) United States
(12) Patent Application Publication
(10) Pub. No.: US 2026/0340779 A1
(43) Pub. Date: Oct. 29, 2026

(54) Method, Apparatus, Device, Medium and Product for Converting MR Image to CT Image Based on Channel and Spatial Attention Generative Adversarial Network

(71) Applicant: Shandong Cancer Hospital and Institute, Jinan City (CN)

(72) Inventors: Zhenjiang LI, Jinan City (CN); Peiping SUN, Jinan City (CN); Baosheng LI, Jinan City (CN); Jian ZHU, Jinan City (CN); Yong YIN, Jinan City (CN)

(21) Appl. No.: 19/378,823

(22) Filed: Nov. 4, 2025

(30) Foreign Application Priority Data
Apr. 29, 2025 (CN) 202510557313.5

(51) Int. Cl. G06T 5/60; G06T 3/4007; G16H 30/40

(52) U.S. Cl. CPC G06T 5/60; G06T 3/4007; G16H 30/40; G06T 2207/10081; G06T 2207/10088; G06T 2207/20081; G06T 2207/20192; G06T 2207/30008

(57) ABSTRACT

A system and method for converting MR image to CT image based on CSAGAN includes constructing a CSAGAN model according to a historic MR image and a corresponding real CT image, where the CSANGAN model includes a generator and a discriminator; the generator includes an encoder, a residual block, a CSA module and a decoder connected in sequence; the discriminator is configured to determine authenticity of a pseudo-CT image according to the real CT image and the pseudo-CT image to determine an output result, and the output result is the pseudo-CT image or the real CT image; and inputting an MR image to be tested into the CSAGAN model, and converting the MR image into the pseudo-CT image as an output. Feature dilution of bones in a deep network can be avoided, collaborative optimization of channel and spatial features is implemented, and edge blurring is avoided.

### CROSS-REFERENCE TO RELATED APPLICATION

This patent application claims the benefit and priority of Chinese Patent Application No. 202510557313.5 filed with the China National Intellectual Property Administration on Apr. 29, 2025, the disclosure of which is incorporated by reference herein in its entirety as part of the present application.

### TECHNICAL FIELD OF THE INVENTION

The present disclosure relates to the field of medical imaging processing, and in particular to a method for converting a single sequence magnetic resonance (MR) image to a computerized tomography (CT) image, an apparatus, a device, a medium, and a product thereof.

### BACKGROUND

Pelvic tumors generally refer to some neoplastic lesions in normal tissues and organs in the pelvic cavity. In female population, common malignant tumors include cervical cancer, ovarian cancer, endometrial cancer, and the like, while male population is more at risk of prostate cancer. In addition, a series of malignant tumors, such as colorectal cancer, bladder cancer and ureteral cancer, may occur in both men and women. It is very important for patients to seek timely standardized diagnosis and treatment in regular hospitals if the patients have some related symptoms. Taking colorectal cancer as an example, if it is found in phase I, its five-year survival rate is in the range of 70%-90%, and if it is not detected until phase II or phase III, the five-year survival rate has already dropped to 50%-70%; once diagnosis is delayed until phase IV, the survival rate has dropped sharply to approximately 20%. With the continuous growth of the global population and the acceleration of aging, the lifestyle and diet structure have changed significantly, which makes the incidence of colorectal cancer increasingly severe. Colorectal cancer has become a major public health problem that cannot be ignored worldwide. At present, in patient radiation therapy, CT images can provide electronic density information, while MR images have excellent soft tissue contrast. To avoid the need for additional CT scans in radiation therapy planning, a series of methods have been proposed to enable MR-only radiation therapy planning. There are problems such as edge blurring and loss of bone details in MR-to-CT conversion in existing methods, such as CycleGAN and Attention U-Net. The conventional model lacks effective fusion of multi-dimensional features, leading to limited quality of generated images. In radiation therapy, CT image is the basis of dose calculation, but has the risk of ionizing radiation, while MR image has no radiation and has high soft tissue contrast, but lacks electron density information.

The existing methods still have the following problems.

The loss of bone details: as pixel-by-pixel loss functions such as mean square error (MSE) are usually used in the conventional generation method, the method tends to generate a fuzzy average result. Bones have a weak signal in the MR image, and the conventional method lacks an explicit attention mechanism to a local high-density region, which leads to gradual dilution of bone features in a deep network. It can be learned from above that in the existing methods, the conventional model is insufficient for feature extraction of high-density bones, resulting in a significant deviation in a Hounsfield Unit (HU) value of a generated synthetic computerized tomography (SCT) image.

Edge blurring: the down-sampling of the conventional generator often employs step-length convolution or pooling operation, accompanied by zero padding to maintain the size. Zero padding may introduce high-frequency artifacts, and it is difficult to fully restore edge information during the up-sampling process in the decoding portion. In addition, when the adversarial training is inadequate, the generator tends to generate a smooth transition rather than a sharp boundary. It can be learned from above that the zero-padding operation in the down-sampling process of the generator in the existing method leads to the blurring of organ boundaries.

Inadequate multi-modal feature fusion: the conventional method usually directly concatenates multi-modal inputs or simply adds feature maps, which lacks dynamic modeling of inter-channel dependence and spatial importance. For example, MR signals of bones and soft tissues are significantly different, but a fixed kernel weight of the conventional convolution cannot adaptively adjust feature weights. It can be learned from above that the existing method lacks collaborative optimization of channel and spatial features.

### SUMMARY OF THE INVENTION

An objective of the present disclosure is to provide a method for converting an MR image to a CT image based on a channel and spatial attention generative adversarial network (CSAGAN), an apparatus, a device, a medium and a product thereof, thereby solving the problems in the prior art.

To achieve the objective described above, the present disclosure provides the following solutions.

In a first aspect, the present disclosure provides a method for converting a magnetic resonance (MR) image to a computerized tomography (CT) image based on a channel and spatial attention generative adversarial network (CSAGAN), including:
constructing a CSAGAN model according to a historic MR image and a corresponding real CT image, where the CSANGAN model includes a generator and a discriminator; the generator includes an encoder, a residual block, a channel and spatial attention (CSA) module and a decoder, which are connected in sequence; the encoder includes a down-sampling module configured to process a feature map of the historic MR image by using reflective padding or learning padding in a down-sampling process to extract high-level semantic features; the residual block is configured to perform deep feature refinement on the high-level semantic features by using a bicubic interpolation up-sampling way to output a feature map; the CSA module is configured to perform channel and spatial weighting on the feature map output by the residual block, highlight a key region and generate a spatial attention map; the decoder is configured to gradually restore a spatial resolution through stridden convolution to be concatenated with the spatial attention map to supplement detail information, and ultimately output a pseudo-CT image; and the discriminator is configured to determine authenticity of the pseudo-CT image according to the real CT image and the pseudo-CT image to determine an output result, where the output result is the pseudo-CT image or the real CT image; and
inputting an MR image to be tested into the CSAGAN model, and converting the MR image to be tested into the pseudo-CT image as an output.

In a second aspect, the present disclosure provides a computer device, including a memory, a processor, and a computer program stored on the memory and run on the processor. The processor is configured to execute the computer program to implement the foregoing method for converting the MR image to the CT image based on the CSAGAN.

In a third aspect, the present disclosure provides a computer readable storage medium, where a computer program is stored on the computer readable storage medium. The computer program, when executed by a processor, is configured to implement the foregoing method for converting the MR image to the CT image based on the CSAGAN.

In a fourth aspect, the present disclosure provides a computer program product, including a computer program, where the computer program, when executed by a processor, is configured to implement the foregoing method for converting the MR image to the CT image based on the CSAGAN.

According to specific embodiments of the present disclosure, the present disclosure has the following technical effects.

A channel and spatial attention generative adversarial network (CSAGAN) model is constructed, and based on a channel and spatial attention (CSA) module in the CSAGAN model, a response to a high-density bone-related feature map is enhanced and an edge is accurately located from channel and spatial dimensions, thereby avoiding feature dilution of bones in a deep network.

Based on the CSA module, the correlation between MR features and target CT features is calculated by combining the channel and spatial dimensions, attention weights are calculated for the spatial and channel dimensions, respectively, and multi-modal information is dynamically fused to implement collaborative optimization of the channel and spatial features.

Finally, during down-sampling, reflective padding or learning padding is used to replace zero padding of the conventional methods to reduce boundary artifacts and preserve local edge consistency. By training to dynamically adapt to boundary features to avoid artificially-induced artifacts, and with the collaboration of the CSA module, direct propagation of underlying bone margin information to the decoder is made, and the introduction of the CSA module can further correct a slight deviation that may remain in the padding.

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without inventive efforts.

FIG. 1 is a flowchart of a method for converting an MR image to a CT image based on a channel and spatial attention generative adversarial network (CSAGAN) according to an embodiment of the present disclosure;

FIG. 2 is a workflow diagram of a CSAGAN model according to an embodiment of the present disclosure;

FIG. 3 is a structural diagram of a CSAGAN model according to an embodiment of the present disclosure;

FIG. 4 is a diagram of a generator for incorporating a CSA module in a CSAGAN model according to an embodiment of the present disclosure;

FIG. 5A-C are diagrams of a CSA module according to an embodiment of the present disclosure, in which FIG. 5A is a diagram of serial combination of a channel attention unit and a spatial attention unit, FIG. 5B is a diagram of the channel attention unit, and FIG. 5C is a diagram of the spatial attention unit;

FIG. 6 is a diagram of a discriminator of a CSAGAN model according to an embodiment of the present disclosure;

FIG. 7A-C are experimental box diagrams for quantitative assessment of a peak signal-to-noise ratio (PSNR) value, a structural similarity index measurement (SSIM) value and a mean absolute error (MAE) value between the model of the present disclosure and other models according to an embodiment of the present disclosure, where the other models include a cycle generative adversarial network (Cycle GAN), a semantic segmentation network of learning target region attention (Attention U-net), a deep convolutional neural network (DCNN), and a registration generative adversarial network (RegGAN), FIG. 7A is an experimental box diagram for quantitative assessment of the PSNR value between the model of the present disclosure and other models, FIG. 7B is an experimental box diagram for quantitative assessment of the SSIM value between the model of the present disclosure and other models, and FIG. 7C is an experimental box diagram for quantitative assessment of the MAE value between the model of the present disclosure and other models;

FIG. 8 is an effect diagram of bone region mask segmentation according to an embodiment of the present disclosure;

FIG. 9 is a structural diagram of an apparatus for converting an MR image to a CT image based on a CSAGAN according to an embodiment of the present disclosure; and

FIG. 10 is a structural diagram of a computer device according to an embodiment of the present disclosure.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. Based on the embodiments of the present disclosure, all other embodiments obtained by those of ordinary skill in the art without inventive efforts shall fall within the protection scope of the present disclosure.

To make the foregoing objectives, features and advantages of the present disclosure more apparent and understandable, the present disclosure is further described in detail with reference to accompanying drawings and specific embodiments.

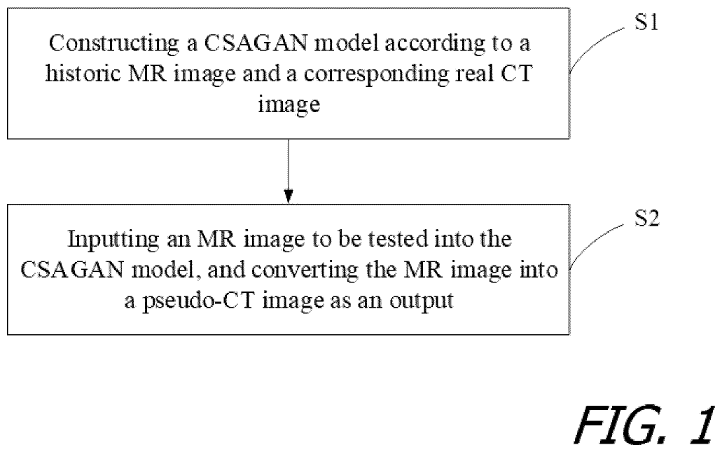

An embodiment of the present disclosure provides a method for converting an MR image to a CT image based on a channel and spatial attention generative adversarial network. The method is executed by a computer device, may specifically be executed by a computer device such as a terminal or a server alone, or may be executed jointly by the terminal and the server. In the embodiment of the present disclosure, as shown in FIG. 1, the method includes the following steps.

S1. A CSAGAN model is constructed according to a historic MR image and a corresponding real CT image, where the CSANGAN model includes a generator and a discriminator. The generator includes an encoder, a residual block, and a CSA module and a decoder, which are connected in sequence. The encoder includes a down-sampling module configured to in a down-sampling process, process a feature map of the historic MR image by using reflective padding or learning padding to extract high-level semantic features. The residual block is configured to perform deep feature refinement on the high-level semantic features by using a bicubic interpolation up-sampling way to output a feature map. The CSA module is configured to perform channel and spatial weighting on the feature map output by the residual block, highlight a key region and generate a spatial attention map. The decoder is configured to gradually restore a spatial resolution through stridden convolution to be concatenated with the spatial attention map to supplement detail information, and ultimately output a pseudo-CT image. The discriminator is configured to determine authenticity of the pseudo-CT image according to the real CT image and the pseudo-CT image to determine an output result, where the output result is the pseudo-CT image or the real CT image.

S2. An MR image to be tested is input into the CSAGAN model, and converted into the pseudo-CT image as an output.

In an exemplary embodiment, prior to S1, the method further includes a preprocessing process.

Data acquisition and preprocessing: registered MR and CT image data are obtained, and then subjected to rigid/non-rigid registration, N4 bias field correction, intensity normalization (an HU value is limited to [−1000, 1000] and normalized to [−1, 1]), and Otsu method for bed artifacts removal, and finally resampled to 256×256 resolution.

In an exemplary embodiment, during construction of the CSAGAN model, the used training data is divided into a training set and a test set in a ratio of 7:3, where the training set includes data of fourteen patients, and the test set includes data of five patients.

The CSGAN model includes the generator G and the discriminator D.

The generator G employs an improved U-Net architecture, where the encoder includes four down-sampling modules, and each down-sampling module includes a maximum pooling layer, a 3×3 convolutional layer, a BatchNorm layer, and a ReLU activation function. The decoder employs bicubic interpolation up-sampling to be concatenated with features of the encoder via skip connection, with nine residual blocks and the CSA module interpolated therein.

The CSA module sequentially executes channel attention and spatial attention operations, where the channel attention can fuse average pooling and maximum pooling features through a shared network (multilayer perceptron, MLP) to generate a channel weight map, and the spatial attention can fuse pooling features on a channel axis through 7×7 convolution to generate a spatial weight map.

The discriminator D is a five-layer fully convolutional network (PatchGAN), each layer includes a convolutional layer with a convolutional kernel of 4×4 and a step size of 2, LeakyReLU activation and instance normalization, and outputs a 256×256 probability matrix with each element corresponding to the authenticity of a local region.

A total loss function Lₜₒₜₐₗ of the CSAGAN model is as follows:

Lₜₒₜₐₗ=Lₐ+L_{c}+Lₛ

where Lₐ is an adversarial loss, that is, a discrimination result of a generated image and a real image are constrained by a mean square error; L_{c} is a cycle consistency loss, that is, a difference between an input image and a reconstructed image is constrained through L1 norm; and Lₛ is a normalized mutual information loss, that is, a structural similarity is calculated through an information entropy and a joint entropy.

$${L}_{s} = {E}_{MR \sim {P}_{( MR )}} [ {‖ \frac{2 \times H ( G ( MR ) , MR )}{H ( G ( MR ) ) + H ( MR )} - 1 ‖}_{1} ]$$

$$H ( MR ) = {\sum }_{{MR}_{i}} [ {p}_{{MR}_{i}} \times {log}_{2} {p}_{( {MR}_{i} )} ]$$

$$H ( G ( MR ) ) = {\sum }_{{( G ( MR ) )}_{j}} [ {p}_{( {( G ( MR ) )}_{j} )} \times {log}_{2} {p}_{( {( G ( MR ) )}_{j} )} ]$$

$$H ( G ( MR ) , MR ) = {\sum }_{{( G ( MR ) )}_{j} , {MR}_{j}} [ {p}_{( {( G ( MR ) )}_{j} {MR}_{i} )} \times {log}_{2} {p}_{( {( G ( MR ) )}_{j} , {MR}_{i} )} ]$$

where G represents the generator for synthesizing sCT, H(MR) is an information entropy of the MR image, H(G(MR)) is information entropy of a sCT image generated by the G, and H(G(MR), MR) is a joint entropy of the input MR image and the generated sCT image. When there is no mapping relationship between the MR image and the generated sCT image, the joint entropy is maximum, Lₛ is 1. When various features of the MR image are mapped to that of the generated sCT image each to each, the joint entropy is minimum, and Lₛ is 0. p_{MR}_{ᵢ} and p_{(G(MR))}_{ᵢ}₎ are HU distributions of the input MR image and the generated sCT image, and p_{((G(MR))}_{ᵢ}_{,MR}_{ᵢ}₎ is a joint distribution of the HU values of the input MR image and the generated sCT image.

The structural consistency is ensured by combining the adversarial loss (the generator is driven to cheat the discriminator, with a formula of mean square error (MSE)), the cycle consistency loss (an L1 distance between the input MR and the reconstructed MR is constrained) and a normalized mutual information constraint (the structural similarity of MR and sCT is calculated through the information entropy).

The CSAGAN model constructed by the present disclosure is a bidirectional conversion model, which not only can convert the MR image into the CT image, but also can convert the CT image into the MR image.

During practical application, training and testing of the CSAGAN model are as follows: Adam optimizer (β₁=0.9, β₂=0.99) is employed, where an initial learning rate is 0.0002, and the model is trained for 400 epochs, with a batch size of 2. During testing, the historic MR image is input to generate an sCT image, namely, the pseudo-CT image.

In an exemplary embodiment, the CSA module includes a channel attention (CA) unit and a spatial attention (SA) unit connected in series.

In the channel attention unit, maximum pooling and average pooling are performed on the feature map output by the residual block to generate a channel attention map, where the channel attention map W_{c}(I) is as follows;

$${W}_{c} ( I ) = ρ ( MLP ( AvgPool ( I ) ) + MLP ( Max Pool ( F ) ) ) = ρ ( {E}_{1} ( {E}_{0} {I}_{avg}^{c} ) ) + {E}_{1} ( {E}_{0} ( {I}_{max}^{c} ) ) ) ,$$

where

$${I}_{avg}^{c} and {I}_{max}^{c}$$

are on average pooling result and a maximum pooling result of the input feature map respectively, E₀ and E₁ are shared weight matrices of MLP, ρ is a Sigmoid activation function, I is the input MR image, and F is feature information of the input MR image.

In the spatial attention unit, the pooling operation is performed along the channel axis of the channel attention map to generate the spatial attention map, where the spatial attention map Wₛ(I) is as follows:

$${W}_{s} ( I ) = ρ ( {f}^{7 \times 7} ( [ AvgPool ( I ) ; Max Pool ( I ) ] ) ) = ρ ( {f}^{7 \times 7} ( [ {I}_{avg}^{s} ; {I}_{max}^{s} ] ) ) ,$$

where

$${I}_{avg}^{s} and {I}_{max}^{s}$$

are an average pooling result and a maximum pooling result along the channel axis respectively, f_{7×7} is convolution operation, and 7×7 is a pixel coverage range of a filter.

In practical application, the channel and spatial attention (CSA) module is as follows: the channel attention unit and the spatial attention unit are connected in series to extract key features from a channel domain and a spatial domain, where the CA unit captures global features through maximum pooling and average pooling to generate the channel weight map and enhance a response of a key channel (such as a bone signal); and the SA unit fuses spatial context information through 7×7 convolution to generate the spatial weight map and fuse on an edge region (such as symphysis pubis).

In practical application, the CA unit specifically includes:
1. A feature map with a size of [B, C, H, W] is input, where B denotes the batch size, C denotes the number of channels, and HOW denotes a spatial size.
2. Branch 1: Maximum pooling branch (Max Pooling Branch).
Step 1: Maximum pooling (Max Pooling): global maximum pooling is performed on the feature map in a spatial dimension.
Step 2: A first convolutional layer is used to compress the number of channels through a Conv2d convolutional operation.
Step 3: A ReLU nonlinear activation function is applied.
Step 4: A second convolution layer is used to restore the number of channels through a Conv2d convolutional operation.
3. Branch 2: Average pooling branch (Avg Pooling Branch).
Step 1: Average pooling (Avg Pooling) is a similar operation to the maximum pooling, and is used for performing global average pooling on the spatial dimension.
Step 2: A first convolutional layer has the same Conv2d convolutional structure as the first convolutional layer of the maximum pooling branch with the same operation.
Step 3: A ReLU nonlinear activation function is applied.
Step 4: A second convolution layer has the same Conv2d convolutional structure as the second convolutional layer of the maximum pooling branch with the same operation to restore the number of channels.
4. Element-wise addition of outputs of the two branches: the final outputs of the two branches are added in the channel dimension, and the added size is the same size as the out size of the two branches.
5. Sigmoid activation function: the foregoing result is processed by Sigmoid to obtain a weight coefficient of each channel with a range of (0, 1), representing the importance of each channel.
6. Output: the attention coefficient is used to perform channel weighting on an original input feature map to enhance a key channel and suppress an unimportant channel.

The SA unit specifically includes the follows.
1. A feature map with a size of [B, C, H, W] is input, where B denotes the batch size, C denotes the number of channels, and H×W denotes the spatial size,
2. Step 1: Maximum pooling of the channel dimension: maximum pooling is performed in the channel dimension (dim=1) to output tensor.
3. Step 2: Mean pooling of the channel dimension: mean pooling is performed in the channel dimension (dim=1) to output the same dimension as that of the maximum pooling.
4. Step 3: Feature Fusion (Concatenation): the two results of maximum pooling and mean pooling are concatenated in the channel dimension to obtain a two-channel spatial attention map.
5. Step 4: Convolutional operation (Conv2d): a Conv2d convolutional operation with a convolutional kernel of 7×7 is applied to the concatenation result to extract spatial attention information.
6. Step 5: Sigmoid activation: an output is mapped to an interval of (0,1) by using Sigmoid function to generate a spatial attention weight map with an output size identical to that of the maximum pooling and the mean pooling.
7. Step 6: The spatial attention map is multiplied with the original input feature map pixel by pixel, and point-by-point weighting is performed through a broadcast mechanism.

In an exemplary embodiment, the down-sampling module includes a maximum pooling layer, a 3×3 convolutional layer, a BatchNorm layer, and a ReLU activation function.

In an exemplary embodiment, the number of residual connections in the residual block is 9, and each residual block includes a residual path branch, and an identity mapping branch.

The residual path branch includes a 0-padding layer, a 3×3 convolutional layer, a BN layer, and a Relu activation function.

The identity mapping branch includes a 1×1 convolutional layer.

In practical application, nine residual blocks are introduced into U-Net to enhance gradient propagation, suppress edge blurring, and alleviate the gradient vanishing problem, while retaining low-level features (such as organ contour).

In an exemplary embodiment, the decoder is connected with the encoder in skip connection, the decoder includes three up-sampling modules, and each up-sampling module includes a stridden convolutional layer, a 3×3 convolutional layer, a BatchNorm layer, and a ReLu activation function, which are connected in sequence.

In an exemplary embodiment, the discriminator is a 5-layer fully convolutional network, each layer of the fully convolutional network includes a convolutional layer with a convolutional kernel of 4×4 and a step size of 2, a LeakyRelu activation function, and an instance normalization layer. Radiation therapy is highly effective in treating malignant tumors such as pelvic tumors. With the development of radiology, radiation therapy techniques have evolved from conventional external beam radiation therapy to 3D conformal radiation therapy (3D-CRT), image guided radiotherapy (IGRT), and intensity-modulated radiotherapy (IMRT). Before implementing the IMRT, a computerized tomography (CT) image of a patient needs to be acquired for planning radiation dose. In addition, a magnetic resonance (MR) image of the corresponding spatial structure is also needed to segment tumor tissues from surrounding normal tissues. MR provides high-contrast soft tissue imaging, while CT provides excellent bone imaging. The principle of CT imaging is based on the difference of X-ray absorptivity of tissues with different densities, which makes the details and textures of bone structures more clearly, and the pixel intensity of the CT image is directly related to electron density information needed for radiation therapy planning. However, the similar densities of various human soft tissues make it difficult for CT to accurately distinguish different soft tissue structures. Moreover, ionizing radiation from CT scans poses potential health risks to the patient, potentially causing secondary complications. Therefore, to avoid the need for additional CT scans in radiation therapy planning, a series of methods have been proposed to enable MR-only radiation therapy planning. One of the effective methods is to synthesize a corresponding CT image from available MR images. By synthesizing the CT image from MR images, radiation therapy planning can obtain more comprehensive information in one image, so that the patient can get more accurate diagnosis and treatment planning.

The technical solution of the present disclosure is further set forth by taking the generation of an sCT image of a pelvic region as an example.

The generation of the sCT image of the pelvic region is as follows.

### 1. Data Preparation:

(1) Data source: 19 cases of pelvic MR and CT images using a Gold Atlas data set.

### (2) Preprocessing:

Registration: rigid/non-rigid registration is performed by using MIM 7.1.9 workstation, with CT as reference.
Correction: N4 bias field correction eliminates the non-uniformity of MR intensity.
Normalization: a HU value of CT is limited to −1000,1000−1000,1000 and linearly normalized to −1, 1−1, 1.
Denoising: Otsu method is used to remove bed shadow artifacts in CT.

(3) Data set division; fourteen cases for training and five cases for testing.

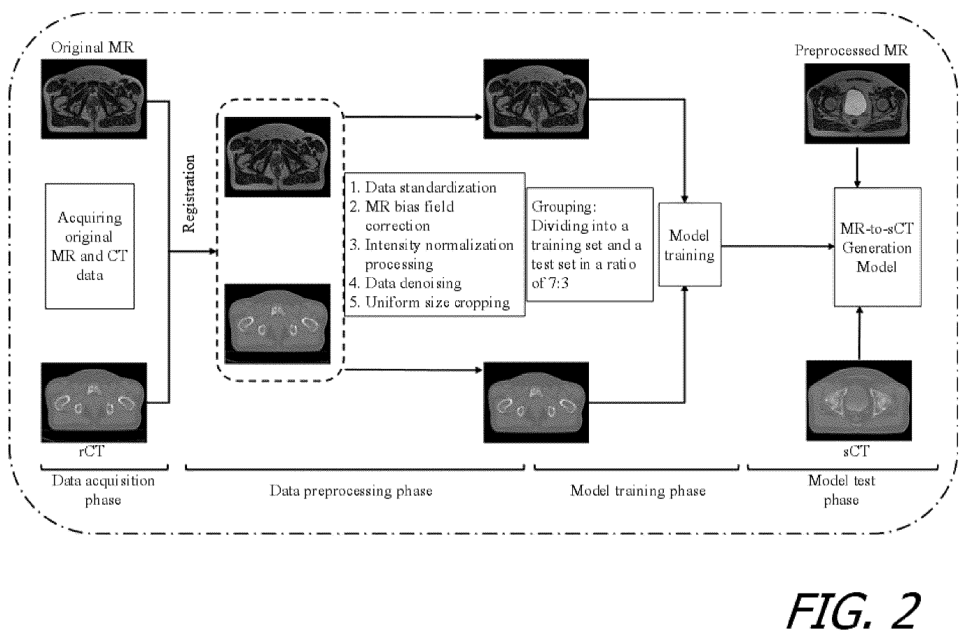

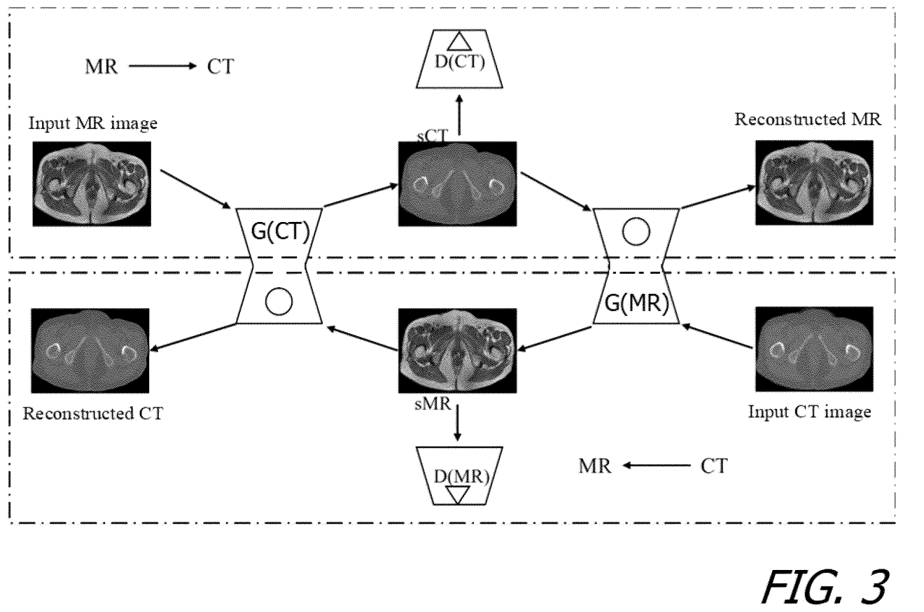

2. As shown in FIG. 2, data preprocessing, model training and testing processes are shown, and the key steps (registration, normalization and CSA module embedding) are labeled. As shown in FIG. 3, the CSAGAN model is constructed as follows.

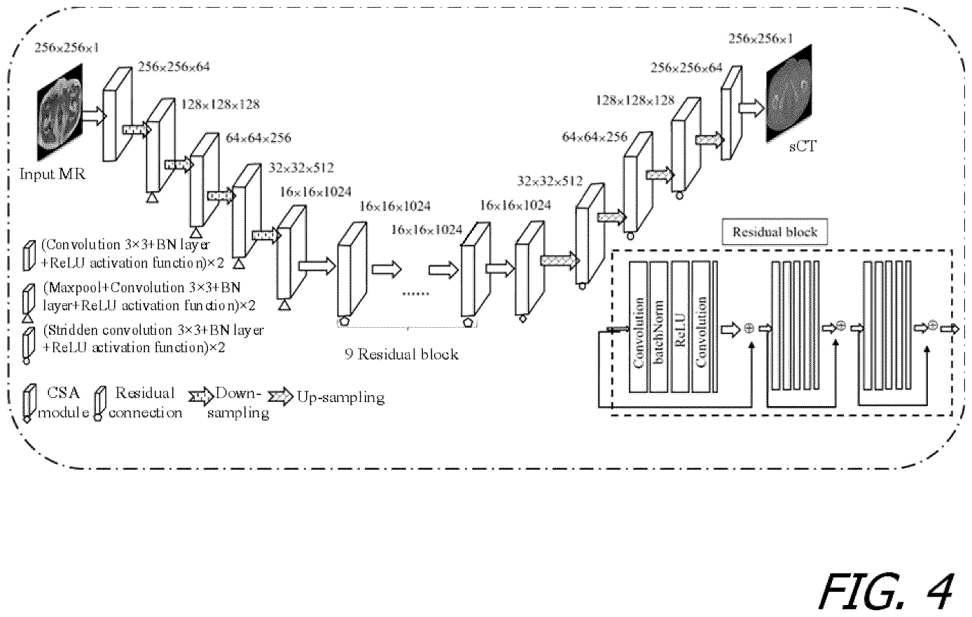

(1) As shown in FIG. 4, the generator G is as follows.

The encoder includes four down-sampling modules, where each down-sampling module includes a maximum pooling layer, two 3×3 convolutional blocks (step size of 1), BatchNorm, and ReLU. Each convolutional block includes (maximum pooling layer, 3×3 convolution, Batch Normalization layer+ReLU activation function)*2.

The decoder employs bicubic interpolation up-sampling to be concatenated with features of the encoder to extract features through 3×3 convolution. A convolutional block structure in the decoder is similar to, but slight differently from, that in the encoder. Each convolutional block of the decoder is also composed of (a convolutional layer, a Batch Normalization layer, and a ReLU activation function)*2. The convolutional layer has a convolutional kernel size of 3×3, a step size of 1, and a padding value of 0.

Residual block: nine residual blocks are provided, each residual block including two 3×3 convolutions (no padding).

FIG. 4 is a specific structure of the generator G (CT) in FIG. 3, which generates the pseudo-CT image from the input MR image, and includes an encoder, a residual module, a CSA attention module, and a decoder. A processed MR is input into the generator, the shallow features of the MR are extracted by the encoder. The residual module solves the problem of gradient vanishing in deep network training and enhances the ability of feature extraction. Then, the CSA attention module performs deep extraction on input features from the channel and spatial dimensions. Finally, the pseudo-CT image is generated through the decoder.

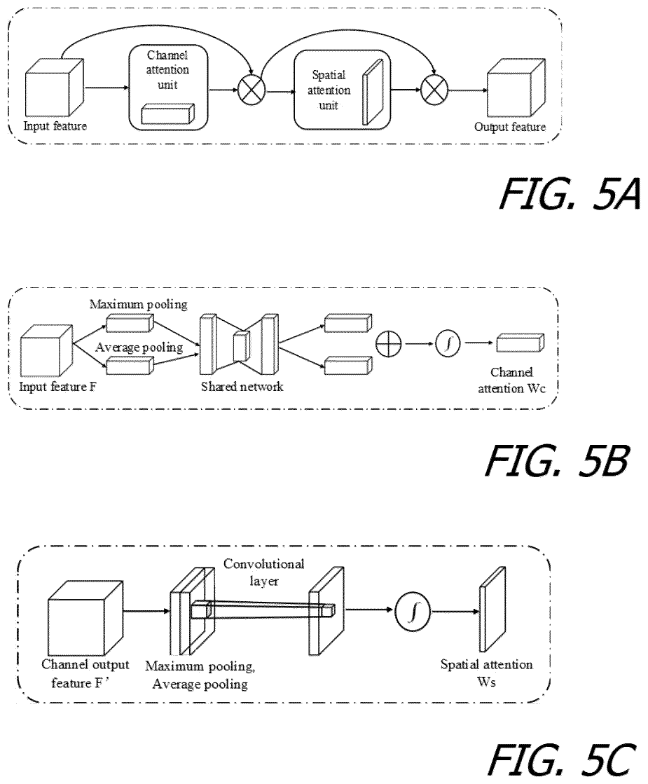

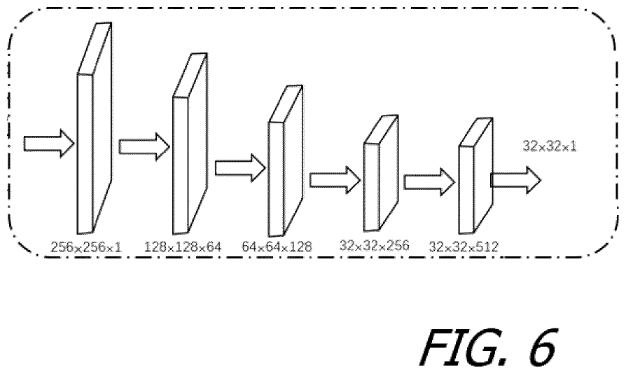

FIG. 6 is a specific structure of the discriminator D (CT) in FIG. 3. An input of the discriminator D is the pseudo-CT image output by the generator in FIG. 3 and the real CT image, and an output of the discriminator D is [0, 1], with the purpose of determining whether the pseudo-CT generated by the generator is similar to the real CT. If the pseudo-CT is similar to the real CT, it is determined as true, and the output value is assigned with 1, and if the pseudo-CT is not similar to the real CT, it is determined as false, and the output value is assigned with 0. FIG. 5(a) is a specific structure of an innovative CSA attention module in the generator structure of FIG. 4, including a channel attention CA module and a spatial attention SA module, which are connected in series.

As shown in the CSA module of FIG. 5A-C, the CSA module is embedded behind the residual block, and performs channel and spatial attention operations in turn. In the channel attention unit, the input feature map is subjected to average pooling and maximum pooling to generate a channel weight through the shared MLP, and is multiplied with original features channel by channel. In the spatial attention unit, the input feature map is pooled and concatenated along the channel axis to generate the spatial weight map through 7×7 convolution, which is multiplied with the original features pixel by pixel.

In an image generation task, it is usually expected to obtain as much global context information and multi-dimensional feature information of the images as possible, while reducing redundant information. However, most existing generative models rarely fuse multi-dimensional feature information. Attention mechanism is an important tool to fuse multi-dimensional feature information. Attention mechanism is that the network learns a set of weight coefficients independently, and then higher weights are assigned to the corresponding feature regions. The common attention mechanisms are channel attention and spatial attention, which are introduced into the CSAGAN model in the present disclosure and called the CSA module.

As shown in FIG. 5(a), the input MR image is represented as I, CSA obtains the channel attention map W_{c} and the spatial attention map Wₛ in turn, that is, FIG. 5(b) and FIG. 5(c), where the whole process can be expressed as follows:

F′=W_{c}(F)⊗I

F″=Wₛ(F′)⊗I′

where ⊗ represents an element-by-element multiplication, and F″ is a final output. The channel sub-module uses a maximum pooling output and an average pooling output of the shared network, and the spatial sub-module uses two similar outputs. The outputs are aggregated along the channel axis and forwarded to the convolutional layer.

The channel attention module generates the channel attention map through the relationship between the channels of the features, and employs the maximum pooling and average pooling to greatly improve expression ability of the network. After the maximum pooling and average pooling operations, the feature information is aggregated to generate two different descriptors: average pooling feature

$${I}_{a v g}^{c}$$

and maximum pooling feature

$${I}_{max}^{c} .$$

generated descriptors are input into the shared network composed of a multi-layer perceptron to output the channel attention map W_{c}∈R^{C×1×1}. Finally, the output features are obtained in a one-by-one summing manner. The channel attention can be calculated as follows:

$${W}_{c} ( I ) = ρ ( MLP ( AvgPool ( I ) ) + MLP ( Max Pool ( F ) ) ) = ρ ( {E}_{1} ( {E}_{0} {I}_{avg}^{c} ) ) + {E}_{1} ( {E}_{0} ( {I}_{max}^{c} ) ) )$$

where ρ is the sigmoid function, E₀∈R^{C/r×C}, and E₁∈R^{C×C/r}. The weights E₀ and E₁ of the shared network are shared for the inputs from the average pooling and the maximum pooling, and E₀ is placed behind the activation function.

In addition, the CSA module aggregates spatial information by using the spatial correlation of feature information to generate the spatial attention map. More feature details are acquired in channel and space, so channel features focus on which information in the image is useful. However, the spatial attention focuses on the location of available information on the feature map. Similarly, in the space attention portion, the average pooling operation and the maximum pooling operation are applied on the channel axis to generate two 2D maps: the average pooling feature

$${I}_{a v g}^{s} ∈ {R}^{1 \times H \times W}$$

and the maximum pooling feature

$${I}_{max}^{s} ∈ {R}^{1 \times H \times W} .$$

A convolutional layer is further applied to generate a two-dimensional spatial attention map Wₛ(I)∈R^{1×H×H×W}, and the spatial attention can be calculated as follows:

$${W}_{s} ( I ) = ρ ( {f}^{7 \times 7} ( [ AvgPool ( I ) ; Max Pool ( I ) ] ) ) = ρ ( {f}^{7 \times 7} ( [ {I}_{avg}^{s} ; {I}_{max}^{s} ] ) )$$

where ρ is the sigmoid function, f^{7×7} represents the convolution operation, and 7×7 represents the pixel coverage range of the filter.

The CSA module according to the present disclosure is a module used between the encoding and decoding portions of the generator network. The feature information is input through the encoder, and attention maps of the input feature information are generated in sequence from the two dimensions of channel and space, and then the feature fusion is carried out effectively. The introduction of this attention mechanism does not increase the burden of model training, but produces better training results.

1. Input, output and cooperation processes of each layer structure are as follows.

(1) Encoder structure includes four down-sampling modules connected in series, each down-sampling module includes: a maximum pooling layer, two 3×3 convolutions (step size of 1, padding of 1)→BatchNorm (batch standardization)→RELU. The down-sampling operation is to reduce the size of the feature map at the end of each down-sampling module through the maximum pooling layer of the next down-sampling module. As shown in Table 1, the size unit of the feature map is PPI (Pixels Per Inch).

TABLE 1
Input/output process schematic table of
input module and down-sampling module

| | Input size (Initial | | The |
|---|---|---|---|
| Module | input is an MR | | number of |
| number | image of 256 × 256) | Output size | channels |
| Input module | 256 × 256 × 1 | 256 × 256 × 64 | 1→ 64 |
| Down-sampling | 256 × 256 × 64 | 128 × 128 × 128 | 64→128 |
| module 1 | | | |
| Down-sampling | 128 × 128 × 128 | 64 × 64 × 256 | 128→256 |
| module 2 | | | |
| Down-sampling | 64 × 64 × 256 | 32 × 32 × 512 | 256→512 |
| module 3 | | | |
| Down-sampling | 32 × 32 × 512 | 16 × 16 × 1024 | 512→1024 |
| module 4 | | | |

The function of the down-sampling module is to extract low-level to high-level features step by step, and reduce spatial resolution and increase the number of channels.

(2) Residual blocks: the structure is formed by connecting nine residual blocks in series, each block includes Branch 1 (residual path): 0 padding→3×3 convolution (step size of 1)→BatchNorm→ReLU→0 padding>3×3 convolution (step size of 1)→BatchNorm; and Branch 2 (identity mapping): if the number of the input channels is inconsistent with that of the output channels, the channels need to be adjusted through 1×1 convolution. Merge: Branch 1 output+Branch 2 input→ReLU. Its function is to relieve gradient vanishing and enhance deep feature expression ability through residual connection.

The input size is 16×16×1024 (output of the encoder), and the output size is 16×16×1024 (the size remains unchanged). Formulation is as follows (taking the first residual block as an example):

xᵢ₊₁=ReLU(F₁(xₗ)+xₗ)

where F₁ is a convolutional operation of Branch 1.

(3) Attention module (Channel+Spatial Attentions): as shown in FIG. 6, the input size is 16×16×1024, and the output size is 16×16×1024 (which is the same as the input). Its function is to enhance the response of important features and suppress redundant information through the channel and spatial attention mechanisms.

(4) Decoder structure includes three up-sampling modules, each module includes stridden convolution (namely, transposed convolution, with a step size of 2)→3×3 convolution (step size of 1)→BatchNorm→ReLU, where skip connection refers to concatenating a feature map output by a corresponding layer of the encoder with a current feature map of the decoder, as shown in Table 2.

TABLE 2
Input/output process schematic table
of CSA module and up-sampling module

| | | Size | Source | |
|---|---|---|---|---|
| Module | | after up- | of skip | Final |
| number | Input size | sampling | connection | output size |
| CSA module | 16 × | 32 × | Output of encoder | 32 × |
| | 16 × 1024 | 32 × 512 | module 3 | 32 × 256 |
| Up-sampling | 32 × | 64 × | Output of encoder | 64 × |
| module 1 | 32 × 512 | 64 × 256 | module 2 | 64 × 128 |
| Up-sampling | 64 × | 128 × | Output of encoder | 128 × |
| module 2 | 64 × 256 | 128 × 128 | module 1 | 128 × 64 |
| Up-sampling | 128 × | 256 × | Output of input | 256 × |
| module 3 | 128 × 128 | 256 × 64 | module | 256 × 32 |
| Output | 256 × | | | 256 × |
| module | 256 × 64 | | | 256 × 1 |

Last layer: 1×1 convolution (step size of 1)→Tanh activation (mapping the pixel value to [−1,1] to adapt to a PNG format). The final output size is 256×256×1 (consistent with the size of the input MR image).

### 2. Summary of Cooperation Flows of all Layers

In the encoder, high-level semantic features of the MR image are progressively extracted through four down-sampling modules (the size is reduced to 16×16×1024), where the feature map output from each layer is stored for subsequent skip connection in the decoder.

The residual block performs deep feature refinement on 16×16×512 features output by the encoder, enhancing nonlinear expression ability through the nine residual blocks.

The attention module performs channel and spatial weighting on the feature map output by the residual block to highlight a key region (such as organ boundary and lesion region).

The decoder progressively restores the spatial resolution by stridden convolution to be concatenated with a feature map of the corresponding layer of the encoder to supplement the detailed information.

Finally, a CT image matching the input size is generated by 1×1 convolution and Tanh activation.

3. A formula of transposed convolution in the up-sampling module is as follows.

Assuming that the input is x∈R^{H×W×C}, a transposed convolution kernel is K∈R^{3×3×C}^{ₒᵤₜ}^{×C}, the step size is 2, and the padding is 1, then the output size is: Hₒᵤₜ=(H−1)×2+3−2×1=2H+1.

An example of an input formula of an i^{th} layer of skip connection in the decoder is as follows:

x_{decoder}=Concat(xᵤₚₛₐₘₚₗₑ,x_{encoder}_{ᵢ}).

(2) The discriminator D is as shown in FIG. 6, that is, D (CT) in FIG. 3.

Structure: a five-layer fully convolutional network, where each layer includes 4×4 convolution (step size of 2), LeakyRelu (x=0.2), and instance normalization.

Output: a 256×256 matrix, where each element represents an authenticity probability for a 70×70 receptive field.

The generator is composed of an encoder (i.e., a shallow feature extraction module), a residual module+CSA attention module (i.e., a deep feature extraction module), and a decoder (i.e., an image synthesis module), with an architecture shown in FIG. 4. Specifically, the encoder shallow feature extraction portion is composed of four convolutional modules and four down-samplings. Each convolutional block includes four portions: (a maximum pooling layer, 3×3 convolution, a Batch Normalization layer, and ReLU activation function)*2, aiming at acquiring detailed local texture information. The deep feature extraction portion employs a structure of residual module+CSA attention module, with the purpose of solving the gradient vanishing problem in deep network training while enhancing feature extraction ability, and acquiring context information with rich semantic information from channel and spatial dimensions. This structure design takes into account that the excessive attention to context information in the shallow portion of the network is meaningless and only increases computational burden on the network. On the contrary, in a deep portion of the network, more multi-dimensional feature descriptions related to the target pixels, rather than just the feature information of fixed local regions, should be considered. This is because the multi-dimensional feature information can provide more context information, which is conducive to describing the target more accurately. In contrast, information expression ability of the fixed local region is limited to some extent.

### (1) Encoder-Shallow Feature Extraction Module

In the encoder shallow feature extraction module, an input image is firstly extended to a size of 256*256*64. Then, when a spatial feature distribution remains unchanged, four convolutional modules and the down-sampling layer are used to implement feature mapping from an image to a high-dimensional space. Each convolutional module is composed of (a maximum pooling layer, 3×3 convolution, a Batch Normalization layer+ReLU activation function)*2. As shown in FIG. 4, the encoder shallow feature extraction module acquires four feature maps with different resolutions through the convolution operation, among which a feature map with the minimum resolution is used as an input of the deep feature extraction module (residual module+CSA attention).

### (2) Residual Module+CSA Attention-Deep Feature Extraction Module

CNN expands the receptive field of the convolution kernel by down-sampling, but the down-sampling process easily leads to the loss of key information and ignores the correlation between the whole and the portions. The deep feature extraction module is composed of a residual module and a CSA attention module. FIG. 4 shows a detailed architecture of the residual block, which is a residual block composed of nine convolutional blocks (each is composed of two 3×3 convolutions, two Batch Normalization layers and a ReLU activation function), in the generator. This design has the following advantages. Firstly, low-level local texture information and context information with rich semantic information are effectively aggregated by the residual connection, which enhances the characterization ability of the network to the image features. Secondly, the residual connection can enhance gradient propagation and improve the stability of the model. Finally, the attention module (CSA) according to the present disclosure can generate attention maps of the input feature information in sequence from channel and spatial dimensions through the feature information input by the residual module, and then effectively perform feature fusion. The introduction of this attention mechanism does not increase the burden of model training, but produces better training effect, making it better approach the feature distribution of the high-resolution image recovered by the image synthesis module, thereby better restoring the details and structure of the high-resolution image.

### (3) Image Synthesis Module

The decoder according to the present disclosure employs a cascaded up-sampler as the image synthesis module, which is composed of multiple bicubic interpolation up-sampling layers and configured to decode hidden features to output a final synthesized image. In the image restoration process, the resolution of the feature map is much less than that of the original image, which inevitably leads to the loss of low-level feature information (such as organ boundaries). The high-level feature information extracted by the up-sampling module is used to help the model better understand the semantic information of the image, thereby producing a more realistic image.

### 4. Parameter Training:

The Adam optimizer (β₁=0.9, β₂=0.99) is employed. An initial learning rate is 0.0002, and is linearly decayed to 0 after 200 epochs.

The batch size is 2.

The training period is 400 epochs.

A specific training optimization process example is as follows:
1) Parameter initialization: a random initialization model weight of model parameters is θ; initialization of Adam state variables: a first-order moment estimation (momentum) m=0, a second-order moment estimation (adaptive learning rate) v=0, and a global iteration step t=0; hyperparameters β₁=0.9 (momentum decay rate), B₂=0.99 (second moment decay rate), ε=1e−8 (numerical stability constant),
2) Learning rate scheduling: the initial learning rateα is 0.0002. A decay strategy is to remain α constant for the first 200 epochs, and then linearly decay α to 0. Specific formula is as follows:

$$α = \{ \begin{matrix}0.0002 & if epoch \leq 200 \\ 0.0002 \times ( 1 - \frac{epoch - 200}{200} ) & otherwise\end{matrix}$$

which is equivalent to α=0.0002×(400−epoch)/200 when 200<epoch≤400.
3) Training cycle (iterated by epoch): for each epoch∈[1, 400], the following procedures are included: (1) a current learning rate is calculated, and α of this current epoch is determined according to the above formula; (2) the data is trained in batches, the training set is divided into multiple batches, each including two samples; and (3) batch-by-batch training is performed according to (2), in which forward propagation is used to calculate the loss of the current batch, and backward propagation is used to calculate the gradient gₜ=∇θ L(θ); the Adam state is updated, and the first-order moment estimation (momentum) formula is as follows:

mₜ=β₁·mₜ₋₁+(1−β₁)·gₜ

the second-order moment estimation (adaptive learning rate) formula is as follows:

vₜ=β₂·vₜ₋₁+(1−β₂)·gₜ²(by element square)

a deviation correction (compensation for initial zero offset) formula is as follows:

$${\hat{m}}_{t} = \frac{{m}_{t}}{1 - {β}_{1}^{t}} , {\hat{v}}_{t} = \frac{{v}_{t}}{1 - {β}_{2}^{t}}$$

where a parameter updating formula is as follows:

$${θ}_{t} = {θ}_{t - 1} - α \cdot \frac{{\hat{m}}_{t}}{\sqrt{{\hat{v}}_{t}} + ϵ}$$

the number of iterative steps increases to t+=1.

### 4) Conclusion of Key Formula.

(1) The learning rate decay formula is as follows (after 200 epoch):

$$α = 0.0002 \times max ( 0 , \frac{400 - epoch}{200} )$$

(2) The Adam parameter updating formula is as follows:

$$θ ← θ - α \cdot \frac{{\hat{m}}_{t}}{\sqrt{{\hat{v}}_{t}} + ϵ}$$

5) Dynamic analysis of training: at an initial phase (epoch≤200), the learning rate is constant (0.0002), and Adam quickly converges by using momentum and adaptive learning rate; at a later phase (epoch >200), the learning rate decays linearly, and a parameter updating extent decreases gradually to avoid oscillation and promote the stable convergence of the model; and batch size=2: increasing gradient noise in small batches may improve generalization, but require more iteration steps.

The model gradually adjusts the parameters through the above process, and finally completes the training within 400 epochs.

The specific training process and experimental environments are as shown in FIG. 3.

TABLE 3
Training process and experimental environment table

| Experimental environment | Version |
|---|---|
| Operating system | Ubuntu 20.04LTS |
| CPU | Intel (R) Core (TM) 19-13900K |
| GPU | NVIDIA Geforce RTX 4090 |
| Memory | 24 G |
| Acceleration environment | CUDA = 11.8, CUDNN = 8.9.2 |
| Pytorch | 2.0.0 |
| Python | 3.9.6 |

### 5. Result Evaluation:

Quantitative index: PSNR 31.83 dB, SSIM 0.913, MAE 42.96 HU, where results are as shown in Table 4.

TABLE 4
Comparison table of quantitative results of sCT generated
by CSAGAN model and comparison models in pelvic region

| Method | PSNR (dB↑) | SSIM (↑) | MAE (HU↓) |
|---|---|---|---|
| Cycle GAN | 29.87 ± 1.06 | 0.898 ± 0.012 | 50.93 ± 4.45 |
| Attention U-Net | 31.56 ± 1.29 | 0.896 ± 0.014 | 47.35 ± 5.16 |
| DCNN | 30.26 ± 1.21 | 0.890 ± 0.018 | 50.16 ± 5.88 |
| RegGAN | 31.64 ± 1.13 | 0.901 ± 0.012 | 44.05 ± 4.98 |
| CSAGAN (ours) | 31.83 ± 0.98 | 0.913 ± 0.018 | 42.96 ± 4.23 |

Qualitative comparison: sCT generated by CSAGAN is closer to the real CT in the pubic margin, bladder contour and other regions, which highlights the details of bone margin.

Ablation experiment: After the CSA module is removed, the MAE increases to 47.96 HU, and the bone details are significantly blurred.

The foregoing evaluation results are significantly superior to those of the comparison models.

The technical solution of the present disclosure is further set forth by taking the optimal generation of bone regions as an example.

### 1) Binary Mask Segmentation:

For a generated binary bone mask, the pelvic bone region is extracted by threshold segmentation (HU>200).

### 2) Loss Function Adjustment:

A weighted MAE loss of the bone region is added to the total loss.

L_{bone}=λ·∥M⊙(G_{CT}(MR)−CT∥₁

where M is a bone mask, and λ=2 is a weight coefficient.

### 3) Effect Verification

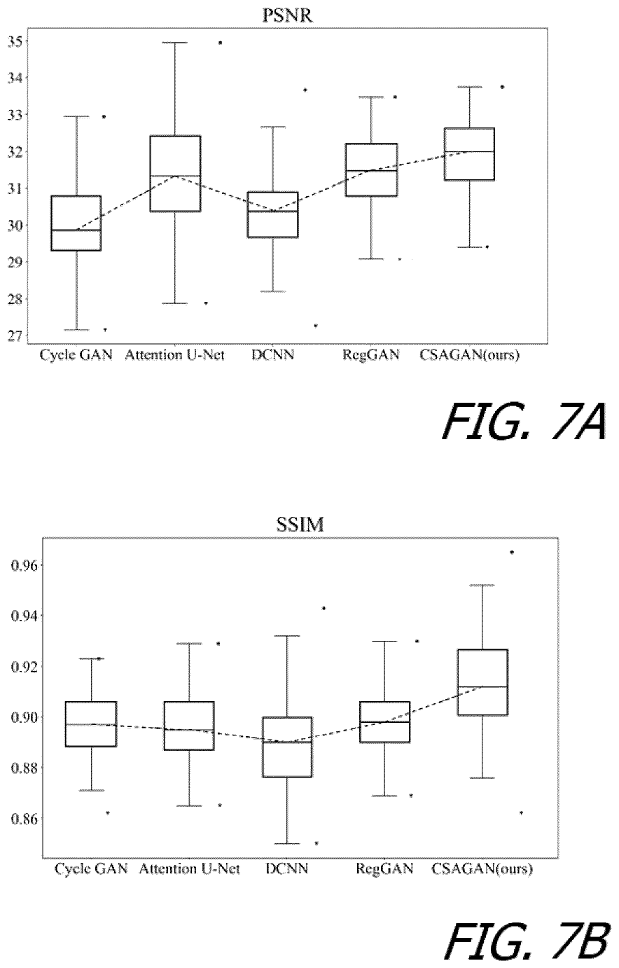

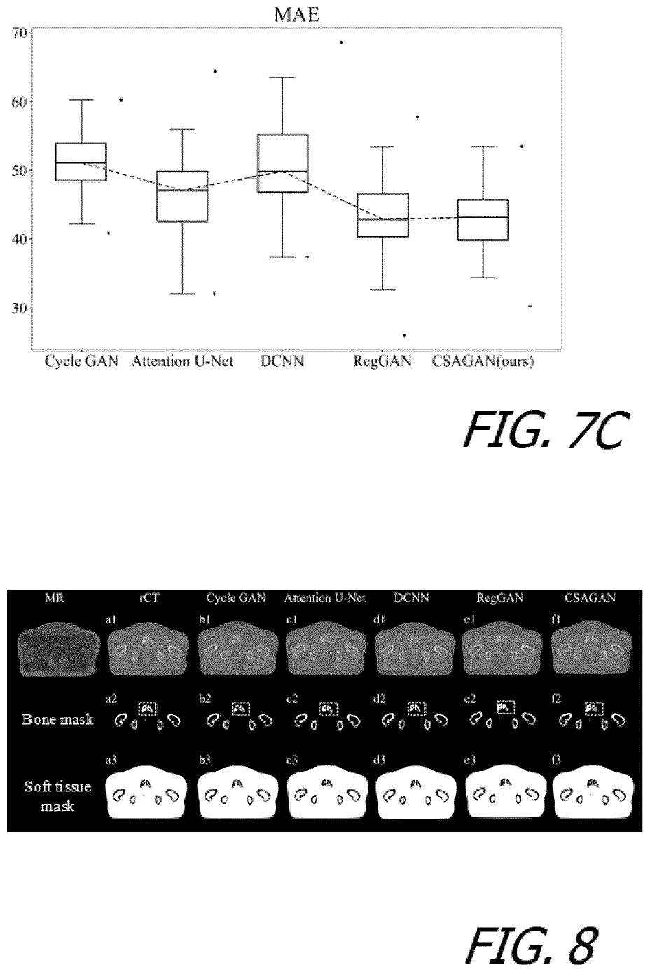

The MAE for the bone region decreases from 137.39 HU (CycleGAN) to 128.36 HU, as shown in Table 5. The clarity of the inferior pubic ramus border is improved by about 30% compared with the worst comparison model, as shown in FIG. 7A-C and FIG. 8.

TABLE 5
Comparison table of quantitative results of sCT generated
by CSAGAN model and comparison models in bone

| Method | PSNR (dB↑) | SSIM (↑) | MAE (HU↓) |
|---|---|---|---|
| Cycle GAN | 30.77 ± 1.56 | 0.891 ± 0.019 | 137.39 ± 26.43 |
| Attention U-Net | 29.56 ± 1.39 | 0.886 ± 0.013 | 138.35 ± 21.36 |
| DCNN | 31.87 ± 1.22 | 0.863 ± 0.025 | 132.17 ± 23.38 |
| RegGAN | 32.96 ± 1.27 | 0.894 ± 0.014 | 134.56 ± 19.87 |
| CSAGAN (ours) | 33.23 ± 1.18 | 0.903 ± 0.016 | 128.36 ± 22.12 |

Implementation effects of the foregoing embodiments are as follows, and the sCT image generated by the present disclosure has the following advantages in clinical radiation therapy plan.
(1) Dose calculation accuracy: the electron density distribution of sCT has an error of less than 3% compared to the real CT.
(2) Multi-organ fidelity: HU errors for soft tissues, such as prostate and rectum, are less than 35 HU.
(3) Calculation efficiency: the generation time of a single 256×256 image is less than 0.5 s, which meets the real-time requirements.

The CSA module according to the present disclosure includes a CA unit and an SA unit connected in series, which are configured to generate multi-dimensional feature weights through maximum pooling, average pooling and 7×7 convolution operation to enhance feature extraction abilities of key regions (such as bones, prostate contour). Residual connection optimization: nine residual blocks are embedded between an encoding layer and a decoding layer of the U-Net generator to avoid edge blurring caused by zero padding and improve gradient propagation stability. Multi-loss joint optimization: combined with adversarial loss (La), cycle consistency loss (Lc) and normalized mutual information constraint loss (Ls), information entropy and joint entropy are calculated to ensure the structural consistency between the generated CT and the input MR. Discriminator design: a fully convolutional PatchGAN structure is employed to determine high-frequency details by a local receptive field, and output a 256×256 probability matrix. The experiment is based on Gold Atlas pelvic data set. The results show that the generated synthesized CT (sCT) according to the present disclosure is better than that of the models, such as CycleGAN and Attention U-Net, in terms of indexes such as PSNR (31.83 dB), SSIM (0.913) and MAE (42.96 HU), and the MAE for the bone region is reduced to 128.36 HU, and the clarity of soft tissue margin is increased by 30%. The present disclosure is suitable for accurate dose calculation and image fusion in radiation therapy planning, which can not only reduce the radiation exposure of patients, but also provide more image information for clinic and assist precision medical treatment.

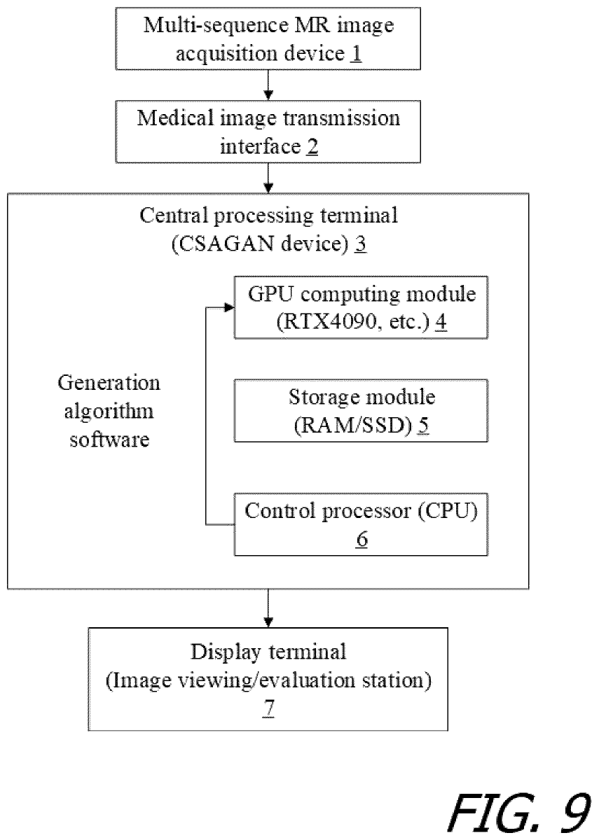

In practical application, the present disclosure further provides an apparatus for converting an MR image to a CT image based on the CSAGAN, which is designed collaboratively based on software and hardware. As shown in FIG. 9, the hardware part of the apparatus includes a multi-sequence MR image acquisition device 1, a medical image transmission interface 2, a central processing terminal 3 and a display terminal 7. The software part of the apparatus includes an image preprocessing module, a CSAGAN generation module, an adversarial discrimination module, and an image output and quality control module.

The apparatus includes: the multi-sequence MR image acquisition device 1, including but not limited to 1.5 T/3.0 T, which can provide original T1/T2 image data, obtain image structure and function information, and correspond to the data input module of the software;
the medical image transmission interface 2, supporting DICOM protocol, which is connected to the processing port, and corresponds to the input/preprocessing module of the software;
the central processing terminal 3 for training and inferring the CSAGAN model, including a GPU computing module 4, including but not limited to RTX4090/A100, which is used for training and inferring the CSAGAN core network (including the encoder, the CSA module and the decoder), and corresponds to the network architecture module (including the generator and the discriminator) of the software; a storage module 5, which is used for scheduling data streams, and storing intermediate features, graphic data, and model weights, and corresponds to the model cache module of the software, where the storage module can be a Random Access Memory (RAM) or a Solid State Drive (SSD); and a control processor 6 (e.g., Central Processing Unit, CPU), which is used for scheduling image reading/calculation/writing, and controlling system flow and quality control checks, and corresponds to the control flow logic and data synchronization module of the software; and
a medical image display terminal station 7, such as a picture archiving and communication system (PACS) station, which is used for displaying the result of generating sCT image for a physician to review and make decisions, and corresponds to the display and quality control module of the software.

During the operation of the exemplary system, an image is first registered, normalized, N4 offset corrected, and resized through the preprocessing module, and then is input into the CSAGAN generation network and processed sequentially through an encoder, multiple residual modules, a channel attention and spatial attention fusion module, and a decoder in order to generate the pseudo-CT image.

The generated image in the embodiment is compared with a real CT image in the discrimination module to optimize the training process and improve the structural accuracy and density restoration ability of the output image.

The exemplary system can complete the inference and output of an image within 0.5 s, and perform clinical usability verification through image quality evaluation metrics, such as PSNR, SSIM, MAE, etc. The final result can be displayed through a DICOM display terminal and uploaded to the hospital PACS system for archiving.

This embodiment can be deployed independently in a server mode, or embedded in the MR terminal to achieve edge inference, and can also be integrated as a doctor station plugin applied in the MR-only radiotherapy workflow, which can ensure image quality and improve automation and operational convenience.

By constructing a hardware-software integrated “CSAGAN image generation system” according to the embodiment, this method can be successfully transformed from a laboratory network model into a clinically applicable device, with the following advantages: GP efficient inference time<0.5 s, meeting real-time clinical requirements; metrics, such as PSNR and SSIM, are superior to various comparison models, and more accurate; the number of CT scans for patients is reduced, so as to lower radiation exposure risks; and software modules are clearly adjustable, and hardware carriers are adapted flexibly.

This embodiment can be deployed as several scenarios, including but not limited to: a medical image processing server in a radiation therapy planning room, an embedded-edge AI device integrated into an MR scanning terminal, a physician station plugin module as a software service system interface, and so on.

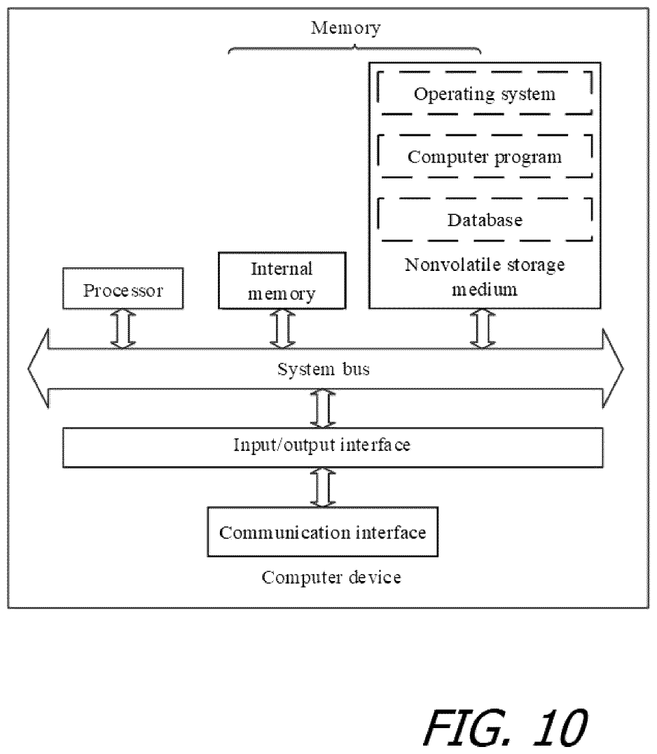

In an exemplary embodiment, a computer device is provided. As shown in FIG. 10, the computer device may be a server or a terminal. The computer device includes a processor, a memory, an input/output interface (abbreviated as I/O), and a communication interface. The processor, the memory and the input/output interface are connected by a system bus, and the communication interface is connected to the system bus through the input/output interface. The processor of the computer device is configured to provide computing and control abilities. The memory of the computer device includes a nonvolatile storage medium and an internal memory. An operating system, a computer program and a database are stored in the nonvolatile storage medium. The internal memory provides an environment for running the operating system and computer program in the nonvolatile storage medium. The database of the computer device is configured to store data on the conversion of an MR image to a CT image of the generative adversarial network based on channel and spatial attentions. The input/output interface of the computer device is configured to exchange information between the processor and an external device. The communication interface of the computer device is configured to communicate with an external terminal through network connection. The computer program, when executed by the processor, is configured to implement the method for converting the MR image to the CT image based on the channel and spatial attention generative adversarial network.

In an exemplary embodiment, a computer device is provided, including a memory and a processor, where a computer program is stored in the memory, and the processor, when executing the computer program, is configured to implement the foregoing method.

In an exemplary embodiment, a computer readable storage medium is provided, on which a computer program is stored. The computer program, when executed by a processor, is configured to implement the foregoing method.

In an exemplary embodiment, a computer program product is provided, and includes a computer program, where the computer program, when executed by a processor, is configured to implement the foregoing method.

Those skilled in the art may understand that all or part of the flows for implementing the foregoing embodiments can be completed by instructing the related hardware through the computer program, which can be stored in the nonvolatile computer-readable storage medium. When executed, the computer program can include the processes of the foregoing embodiments. Any reference to memory, database or other media used in the embodiments according to the present disclosure may include at least one of non-volatile and volatile memories. The nonvolatile memory may include a read only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, and the like. The volatile memory may include a random access memory (RAM), or an external cache memory. By way of illustration than limitation, RAM may be in various forms, such as a static random access memory (SRAM) or a dynamic random access memory (DRAM).

In the present disclosure, all actions to acquire signals, information or data are carried out under the premise of complying with the corresponding data protection laws and policies of the local country and obtaining authorization from the corresponding apparatus owner.

The database involved in individual embodiments according to the present disclosure may include at least one of a relational database and a non-relational database. The non-relational database may include, but is not limited to, a distributed database based on a block chain. The processors involved in the embodiments according to the present disclosure may be general-purpose processors, central processing units, graphics processing units, digital signal processors, programmable logics, data processing logics based on quantum computing, and the like, without limitation.

Technical features of the foregoing embodiments may be randomly combined. To make description concise, not all possible combinations of the various technical features in the foregoing embodiments are described. However, the combinations of these technical features shall be considered as falling within the scope recorded by this specification provided that no conflict exists.

Several specific examples are used for illustration of the principles and implementations of the present disclosure. The description of the embodiments is merely used to help illustrate the method and core principles of the present disclosure. In addition, those of ordinary skill in the art can make various modifications in terms of specific implementations and application scope in accordance with the teachings of the present disclosure. In conclusion, the content of this specification shall not be construed as a limitation to the present disclosure.

What is claimed is:

1. A method for converting a magnetic resonance (MR) image to a computerized tomography (CT) image, said method comprising:
obtaining a historic MR image of an area of interest, wherein said historic MR image contains a first feature map;
conducting a CT scan of said area of interest to obtain a real CT image;
providing a channel and spatial attention generative adversarial network (CSAGAN) model having a generator and a discriminator,
wherein said generator includes an encoder, a residual block, a channel and spatial attention (CSA) module, and a decoder, connected in sequence;
wherein said encoder has a down-sampling module configured to process said first feature map of said historic MR image by using a down-sampling process that includes a group comprising reflective padding and learning padding to extract high-level semantic features;
wherein said residual block conducts deep feature refinement on said high-level semantic features by using a bicubic interpolation up-sampling to produce a second feature map;
wherein said CSA module performs channel and spatial weighting on said second feature map, highlights a key region, and generates a spatial attention map;
wherein said decoder restores a spatial resolution through stridden convolution to be concatenated with said spatial attention map to supplement detail information and output a pseudo-CT image;
wherein said discriminator determines an authenticity of said pseudo-CT image according to said real CT image and said pseudo-CT image to determine an output result which is selected from said pseudo-CT image and said real CT image; and
inputting a new MR image to be tested into said CSAGAN model, and converting said new MR image into a new pseudo-CT image as an output.

2. The method according to claim 1, wherein said CSAGAN model includes a total loss function Lₜₒₜₐₗ where
Lₜₒₜₐₗ=Lₐ+L_{c}+Lₛ
wherein Lₐ is an adversarial loss, L_{c} is a cycle consistency loss, and Lₛ is a normalized mutual information loss.

3. The method according to claim 1, wherein said CSA module comprises a channel attention unit and a spatial attention unit connected in series, wherein said channel attention unit generates a channel attention map W_{c}(I) by performing maximum pooling and average pooling on said second feature map output by said residual block, wherein said channel attention map W_{c}(I) is as follows:
$${W}_{c} ( I ) = ρ ( MLP ( AvgPool ( I ) ) + MLP ( Max Pool ( F ) ) ) = ρ ( {E}_{1} ( {E}_{0} ( {I}_{avg}^{c} ) ) + {E}_{1} ( {E}_{0} ( {I}_{max}^{c} ) ) )$$
wherein
$${I}_{avg}^{c} and {I}_{max}^{c}$$
are an average pooling result and a maximum pooling result of an input feature map respectively, E₀ and E₁ are shared weight matrices of MLP (Multilayer Perceptron), ρ is a Sigmoid activation function, I is an input MR image, and Fis feature information of said input MR image;
in said spatial attention unit, a pooling operation is performed along a channel axis of said channel attention map to generate said spatial attention map, wherein said spatial attention map Wₛ(I) is as follows:
$${W}_{s} ( I ) = ρ ( {f}^{7 \times 7} ( [ AvgPool ( I ) ; Max Pool ( I ) ] ) ) = ρ ( {f}^{7 \times 7} ( [ {I}_{avg}^{s} ; {I}_{max}^{s} ] ) ) )$$
wherein
$${I}_{avg}^{s} and {I}_{max}^{s}$$
are an average pooling result and a maximum pooling result along said channel axis respectively, f^{7×7} is a convolution operation, and 7×7 is a pixel coverage range of a filter.

4. The method according to claim 1, wherein said down-sampling module comprises a maximum pooling layer, a 3×3 convolutional layer, a BatchNorm layer, and a ReLU activation function;
wherein said residual block comprises nine residual connections, and each of said residual connections comprises a residual path branch and an identity mapping branch;
wherein said residual path branch comprises a 0-padding layer, a 3×3 convolutional layer, a BN layer, and a ReLU activation function; and
wherein said identity mapping branch comprises a 1×1 convolutional layer;
wherein said decoder and said encoder are in a skip connection, said decoder comprises three up-sampling modules, and each of said up-sampling modules comprise a stridden convolutional layer, a 3×3 convolutional layer, a BatchNorm layer, and a ReLu activation function connected in sequence; and
said discriminator is a fully convolutional network with five layers, wherein each of said layers comprises a convolutional layer with a convolutional kernel of 4×4 and a step size of 2, a LeakyReLU activation function, and an instance normalization layer.

5. An apparatus for converting a magnetic resonance (MR) image to a computerized tomography (CT) image based on a channel and spatial attention generative adversarial network (CSAGAN), comprising:
a multi-sequence MR image acquisition device for acquiring image data, wherein said image data comprises a historic MR image and a corresponding real CT image;
a medical image transmission interface for transmitting said image data to a central processing terminal; and
wherein said central processing terminal constructs a CSAGAN model according to said historic MR image and said corresponding real CT image, inputting a new MR image to be tested into said CSAGAN model, and converting said new MR image to be tested into a pseudo-CT image as an output.

6. The apparatus according to claim 5, wherein said central processing terminal comprises a GPU computing module, a storage module and a control processor;
wherein said GPU computing module is configured for training and inferring an encoder, a channel and spatial attention (CSA) module, and a decoder in said CSAGAN model;
wherein said storage module is configured for scheduling data streams, and storing intermediate features, graphic data, and model weights; and
wherein said control processor is configured for scheduling image reading, calculation and writing, and controlling an overall system flow and quality control checks.

7. The apparatus according to claim 5, further comprising a display terminal configured for displaying said pseudo-CT image, wherein said display terminal is a medical image display terminal.

8. A computer device, comprising:
a memory,
a processor that runs a computer program stored on said memory, wherein said processor produces said (CSAGAN) model for converting the MR image to the CT image based on the method according to claim 1.

9. The computer device according to claim 8, wherein said CSAGAN model has a total loss function Lₜₒₜₐₗ as follows:
Lₜₒₜₐₗ=Lₐ+L_{c}+Lₛ
wherein Lₐ is an adversarial loss, L_{c} is a cycle consistency loss, and Lₛ is a normalized mutual information loss.

10. The computer device according to claim 8, wherein said CSAGAN model has a generator and a discriminator, and said generator includes an encoder, a residual block, a channel and spatial attention (CSA) module, and a decoder, connected in sequence;
wherein said CSA module comprises a channel attention unit and a spatial attention unit connected in series;
wherein said channel attention unit generates a channel attention map by performing maximum pooling and average pooling on a feature map output of said residual block, wherein said channel attention map W_{c}(I) is as follows:
$${W}_{c} ( I ) = ρ ( MLP ( AvgPool ( I ) ) + MLP ( Max Pool ( F ) ) ) = ρ ( {E}_{1} ( {E}_{0} ( {I}_{avg}^{c} ) ) + {E}_{1} ( {E}_{0} ( {I}_{max}^{c} ) ) )$$
wherein
$${I}_{avg}^{c} and {I}_{max}^{c}$$
are an average pooling result and a maximum pooling result of an input feature map respectively, E₀ and E₁ are shared weight matrices of MLP (Multilayer Perceptron), ρ is a Sigmoid activation function, I is an input MR image, and Fis feature information of said input MR image;
wherein said spatial attention unit performs a pooling operation along a channel axis of said channel attention map to generate said spatial attention map, wherein said spatial attention map Wₛ(I) is as follows:
$${W}_{s} ( I ) = ρ ( {f}^{7 \times 7} ( [ AvgPool ( I ) ; Max Pool ( I ) ] ) ) = ρ ( {f}^{7 \times 7} ( [ {I}_{avg}^{s} ; {I}_{max}^{s} ] ) )$$
wherein
$${I}_{avg}^{s} and {I}_{max}^{s}$$
are an average pooling result and a maximum pooling result along said channel axis respectively, f^{7×7} is a convolution operation, and 7×7 is a pixel coverage range of a filter.

11. The computer device according to claim 10, wherein said encoder has a down-sampling module that includes a maximum pooling layer, a 3×3 convolutional layer, a BatchNorm layer, and a ReLU activation function;
said residual block comprises nine residual connections, and each residual block comprises a residual path branch, and an identity mapping branch;
wherein said residual path branch comprises a 0-padding layer, a 3×3 convolutional layer, a BN layer, and a ReLU activation function; and
wherein said identity mapping branch comprises a 1×1 convolutional layer;
wherein said decoder and said encoder are in skip connection, said decoder having three up-sampling modules, and each of said up-sampling modules comprise a stridden convolutional layer, a 3×3 convolutional layer, a BatchNorm layer, and a ReLu activation function connected in sequence; and
said discriminator is a fully convolutional network with five layers wherein each of said layers includes a convolutional layer with a convolutional kernel of 4×4 and a step size of 2, a LeakyReLU activation function, and an instance normalization layer.

12. A computer program product, comprising a computer program, wherein the computer program, when executed by a processor, produces the CSAGAN model according to claim 1.

13. The computer program product according to claim 12, wherein said CSAGAN model has a total loss function Lₜₒₜₐₗ where:
Lₜₒₜₐₗ=Lₐ+L_{c}+Lₛ
wherein Lₐ is an adversarial loss, L_{c} is a cycle consistency loss, and Lₛ is a normalized mutual information loss.

14. The computer program product according to claim 12, wherein said CSAGAN model has a generator and a discriminator, and said generator includes an encoder, a residual block, a channel and spatial attention (CSA) module, and a decoder, connected in sequence;
wherein said CSA module comprises a channel attention unit and a spatial attention unit connected in series;
wherein said channel attention unit generates a channel attention map by performing maximum pooling and average pooling on a feature map output by said residual block, wherein said channel attention map W_{c}(I) is as follows:
$${W}_{c} ( I ) = ρ ( MLP ( AvgPool ( I ) ) + MLP ( Max Pool ( F ) ) ) = ρ ( {E}_{1} ( {E}_{0} ( {I}_{avg}^{c} ) ) + {E}_{1} ( {E}_{0} ( {I}_{max}^{c} ) ) )$$
wherein
$${I}_{avg}^{c} and {I}_{max}^{c}$$
are an average pooling result and a maximum pooling result of an input feature map respectively, E₀ and E₁ are shared weight matrices of MLP (Multilayer Perceptron), ρ is a Sigmoid activation function, I is an input MR image, and F is feature information of said input MR image;
wherein said spatial attention unit performs a pooling operation along a channel axis of said channel attention map to generate said spatial attention map, wherein said spatial attention map Wₛ(I) is as follows:
$${W}_{s} ( I ) = ρ ( {f}^{7 \times 7} ( [ AvgPool ( I ) ; Max Pool ( I ) ] ) ) = ρ ( {f}^{7 \times 7} ( [ {I}_{avg}^{s} ; {I}_{max}^{s} ] ) )$$
wherein
$${I}_{avg}^{s} and {I}_{max}^{s}$$
are an average pooling result and a maximum pooling result along said channel axis respectively, f^{7×7} is a convolution operation, and 7×7 is a pixel coverage range of a filter.

15. The computer program product according to claim 14, wherein said encoder has a down-sampling module that includes a maximum pooling layer, a 3×3 convolutional layer, a BatchNorm layer, and a ReLU activation function;
said down-sampling module having a maximum pooling layer, a 3×3 convolutional layer, a BatchNorm layer, and a ReLU activation function;
wherein said residual block comprises nine residual connections, and each residual block comprises a residual path branch, and an identity mapping branch;
wherein said residual path branch comprises a 0-padding layer, a 3×3 convolutional layer, a BN layer, and a ReLU activation function; and
wherein said identity mapping branch comprises a 1×1 convolutional layer;
said decoder and said encoder are in skip connection, said decoder having three up-sampling modules, and each up-sampling module comprises a stridden convolutional layer, a 3×3 convolutional layer, a BatchNorm layer, and a Relu activation function connected in sequence; and
wherein said discriminator is a fully convolutional network with five layers, wherein each of said layers has a convolutional layer with a convolutional kernel of 4×4 and a step size of 2, a LeakyReLU activation function, and an instance normalization layer.
